(19) United States
(12) Patent Application Publication
(10) Pub. No.: US 2024/0050423 A1
(43) Pub. Date: Feb. 15, 2024

(54) USE OF PRIDOPIDINE FOR THE TREATMENT OF ANXIETY AND DEPRESSION

(71) Applicant: Prilenia Neurotherapeutics Ltd., Yakum (IL)

(72) Inventors: Michael HAYDEN, Yakum (IL); Mahmoud Abdulhossein Pouladi, Singapore (SG)

(73) Assignee: Prilenia Neurotherapeutics Ltd., Yakum (IL)

(21) Appl. No.: 18/489,863

(22) Filed: Oct. 19, 2023

Related U.S. Application Data
(62) Division of application No. 17/074,901, filed on Oct. 20, 2020.
Application No. 17/074,901 is a continuation-in-part of application No. 16/332,366, filed on Mar. 12, 2019, now abandoned.
(60) Provisional application No. 62/532,728, filed on Jul. 14, 2017.
(60) Provisional application No. 62/395,259, filed on Sep. 15, 2016.

(51) Int. Cl. A61K 31/4453; A61K 9/00; A61P 25/22; A61K 9/48; A61P 25/24

(52) U.S. Cl. CPC A61K 31/4453; A61K 9/0053; A61P 25/22; A61K 9/48; A61P 25/24

(57) ABSTRACT

The invention provides a method of reducing anxiety and/or depression in a subject comprising periodically administering to the subject a pharmaceutical composition comprising an amount of pridopidine effective to reduce anxiety and/or depression in a subject.

### CROSS REFERENCE

This application is a Divisional Application from U.S. application Ser. No. 17/074,901 filed Oct. 20, 2020, which is a Continuation-in-Part Application from U.S. application Ser. No. 16/332,366 filed Mar. 12, 2019, which is a National Phase Application from International Application No. PCT/US2017/051814 filed 15 Sep. 2017 which claims the benefit of U.S. Provisional Application No. 62/532,728, filed Jul. 14, 2017 and U.S. Provisional Application No. 62/395,259, filed Sep. 15, 2016, the contents of each of which are hereby incorporated by reference in their entirety.

Throughout this application various publications are referenced, most typically by the last name of the first author and the year of publication. Full citations for these publications are set forth in a section entitled References immediately preceding the claims. The disclosures of all referenced publications in their entireties are hereby incorporated by reference into this application in order to more fully describe the state of the art to which the invention relates.

### BACKGROUND

### Pridopidine

Pridopidine (4-[3-(methylsulfonyl)phenyl]-1-propyl-piperidine) (formerly known as ACR16) is a drug under development for treatment of Huntington disease. The chemical name of pridopidine is 4-(3-(Methylsulfonyl)phenyl)-1-propylpiperidine and its Chemical Registry Number is CAS 346688-38-8 (CSID: 7971505, 2016). The Chemical Registry number of pridopidine hydrochloride is 882737-42-0 (CSID: 25948790 2016).

Pridopidine is a highly selective Sigma-1 receptor (S1R) ligand which has ˜30-fold higher affinity towards the S1R vs D3Rs, and ˜500-fold higher affinity vs D2Rs. Selective binding of pridopidine for the S1R was confirmed using positron emission tomography (PET) imaging in rats (Sahlholm, 2015), and in humans at dose of 45 mg BID (Grachev, 2020). Pridopidine exerts neuroprotective properties which are mediated by its activation of the S1R, as its silencing by genetic or pharmacological methods abolishes the protective effects (Geva 2016, Eddings 2019, Ryskamp 2018, Ionescu 2019).

The S1R is a highly conserved transmembrane protein located in the endoplasmic reticulum (ER) and specifically enriched in the subregions contacting mitochondria (Mitochondria-Associated Membranes, MAM). The S1R is highly enriched in the CNS and specifically within the Basal Ganglia, cortex, and brainstem. The S1R is implicated in cellular differentiation, neuroplasticity, neuroprotection and cognitive function in the brain. Activation of the S1R by pridopidine results in the induction of several cellular processes that are altered in neurodegenerative diseases and neuronal disorders, and their activation contributes to neuroprotection.

Recent transcriptomic analysis of rat striatum showed that pridopidine treatment activates expression of the BDNF, glucocorticoid receptor (GR), and the serine-threonine kinase protein kinase B (Akt)/phosphoinositide 3-kinase (PI3K) pathways, known to promote neuronal plasticity and survival (Geva 2016). Pridopidine enhances the secretion of the neuroprotective brain-derived neurotrophic factor (BDNF) in a neuroblastoma cell line, in a S1R-dependent manner (Geva 2016). Pridopidine rescues the decrease in BDNF levels in neurodegenerative mouse models of HD and Parkinson's Disease, and activates downstream intracellular signaling pathways (Squitieri 2015, Francardo 2019). Thus, modulation of the BDNF pathway is a major component of pridopidine's S1R-mediated neuroprotective effects. BDNF is an important regulator of synaptic plasticity, and both neurotrophins and abnormal plasticity processes are associated with depression.

Dendritic spines facilitate synaptic transmission and plasticity, integrating physiological and morphological changes. A decrease in dendritic spine density in the hippocampus is associated with anxiety and depressive states (Qiao 2015). Pridopidine treatment increased spine density in medium spiny neurons from HD YAC128 mice in a S1R-mediated manner (Ryskamp 2017).

One type of plasticity regulated by BDNF is homeostatic synaptic plasticity (HSP), the processes that maintain the stability of neuronal networks and underlie learning and cognitive capabilities (Smith-Dijak et al., 2019). Homeostatic plasticity is disrupted in major depressive disorder (MDD), and its activation has been suggested as a possible therapeutic avenue (Workman et al., 2017). Pridopidine treatment rescued the impaired HSP observed in cultured cortical neurons from HD YAC128 model mice (Smith-Dijak et al., 2019).

The default-mode network (DMN) is a set of brain regions which are active when the brain is not engaged in a cognitive task, and which are deactivated upon cognitive engagement with a task. Alterations in DMN connectivity and function are associated with MDD and anxiety, and are normalized upon treatment with anti-depressants (Yan 2019; Couthino 2016; Posner 2013). Pridopidine treatment has been shown to increase DMN activity in healthy volunteers and in HD patients.

### SUMMARY OF THE INVENTION

In one aspect, provided herein is a method of reducing anxiety and/or depression in a subject in need thereof comprising periodically administering to the subject a pharmaceutical composition comprising an amount of pridopidine effective to reduce anxiety and/or depression in a subject.

In some embodiments, the method reduces anxiety in the subject. In some embodiments, the method reduces depression in the subject.

Further provided is pridopidine for use in reducing anxiety and/or depression in a subject. Also provided is pridopidine for the manufacture of a medicament for use in reducing anxiety and/or depression in a subject.

Further provided is a pharmaceutical composition comprising an effective amount of pridopidine for reducing anxiety and/or depression in a subject. Also provided is a pharmaceutical composition comprising pridopidine for use in reducing anxiety and/or depression a subject.

The present invention further provides a package comprising:
a) a pharmaceutical composition comprising an amount of pridopidine; and
b) instructions for use of the pharmaceutical composition to reduce anxiety and/or depression in a subject.

In another aspect the present invention provides, a therapeutic package for dispensing to, or for use in dispensing to, a subject, which comprises:
a) one or more unit doses, each such unit dose comprising an amount of pridopidine thereof, wherein the amount of said pridopidine in said unit dose is effective, upon administration to said subject, to reduce anxiety and/or depression in the subject, and
b) a finished pharmaceutical container therefor, said container containing said unit dose or unit doses, said container further containing or comprising labeling directing the use of said package in the reduction of anxiety and/or depression in said subject.

### BRIEF DESCRIPTION OF THE FIGURES

FIGS. 1-4—The effect of pridopidine on anxiety and depression-like behaviors in the YAC128 mouse model of HD.

FIG. 1. Pridopidine administration. The timeline shows the early and late administration times of pridopidine relative to molecular, neuroanatomical and behavioural phenotypes in the rodent model of Huntington Disease, the YAC128 mice. The molecular and neuroanatomical phenotypes are listed along the upper horizontal line. The behavioural phenotypes are listed along the lower horizontal line. Age of the mice, in months, is presented between the upper and lower lines.

FIGS. 2A and 2B. Early and late treatment study designs. (2A) Late pridopidine treatment study design. WT mice were administered vehicle (ddH2O) only, whereas YAC128 HD mice were administered either vehicle (ddH2O) or an escalating dose of pridopidine (10 mg/kg in week 1, 20 mg/kg in week 2, and 30 mg/kg in weeks 3-8). Treatment started at 8 months of age (manifest) and continued for 2 months. (2B) Early pridopidine treatment study design. WT mice were administered vehicle (ddH2O) only, whereas YAC128 HD mice were administered either vehicle (ddH2O) or pridopidine (30 mg/kg). Treatment started at 1.5 months of age (pre-manifest) and continued for 10.5 months. For both (2A) and (2B) behavioural tests were carried out as indicated: OF=open field, EPM=elevated plus maze, FST=forced swim test.

FIG. 3: Pridopidine improves depressive-like behavior in late-stage (manifest) pridopidine treated YAC128 mice in the forced swim test YAC128 HD mice displayed increased depressive-like behavior compared to WT mice. Pridopidine (30 mg/kg) significantly improves depressive-like phenotype of YAC128 HD mice in the forced swim test by reducing immobility time in the water (FIG. 3). Values shown as mean±SEM; n=4 (M) WT-vehicle, n=8 (M) YAC128-vehicle, n=9 (M), n=8 YAC128-pridopidine; *p<0.05 by one-way ANOVA with Fisher's LSD post hoc analysis.

FIGS. 4A-4C. Pridopidine treatment improves Affective functions in early-stage (pre-manifest) treated mice. FIG. 4A (Open field), 4B (Elevated plus maze) and 4C (Forced swim test). YAC128 HD mice displayed increased anxiety-like behavior in the open field at 6 months (A) and elevated plus maze at 8 months of age (B) compared to WT mice. Early pridopidine treatment improves anxiety- and depressive-like phenotypes in YAC128 HD mice. Pridopidine 30 mg/kg increased the time spent in the center of the arena (4A) and in the open arms (4B), indicating decreased anxiety-like behavior. Vehicle-treated YAC128 HD mice showed a trend towards an increased time spent immobile compared with vehicle-treated WT mice in the forced swim test, while pridopidine treatment reduced the time spent immobile, indicating that pridopidine reduces depressive-like behavior (4C). Veh=Vehicle; Pri=Pridopidine; M=males. Values shown as mean±SEM; n=4 (M) WT-vehicle, n=8 (M) YAC128-vehicle, n=9 (M), n=8 YAC128-pridopidine; *p<0.05, **p<0.01, ***p<0.001 by one-way ANOVA with Fisher's LSD post hoc analysis; ##p<0.01 by paired Students t-test.

FIGS. 5A-5B present the study design of rat Forced Swim Test (FIG. 5A) and anti-depressive effect of pridopidine (FIG. 5B) in the Rat Forced Swim. 5A: rats were placed in the swimming tank for a swimming session on day one, and then treated daily with pridopidine, either 3 or 15 mg/kg by oral gavage (per os, po) for 7 days. On day 8, rats were again placed in a water tank, and time spent immobile measured. FIG. 5B: Pridopidine treatment reduces the percent of time spent immobile in the tank, at both 3 mg/kg (non-significant) and 15 mg/kg (significant) indicating an anti-depressive effect.

FIG. 6 Anxiolytic effect of pridopidine in the marble burying test in mice (NS). Object burying is indicative of anxiety in rodents. Inhibition of object-burying in rodents is an accepted model for measuring anxiolytic effects of a drug (Broekkamp et al, 1986; Treit, 1985; Treit et al, 1981). Marble burying activity was assessed in male NMRI mice, which were then treated with pridopidine 1, 3, 10 or 30 mg/kg for 30 minutes before being tested again for marble burying. Pridopidine shows a dose-dependent inhibition of marble burying, indicating an anxiolytic effect. Marble burying also represents an animal model for Obsessive-compulsive disorder.

Data shown are mean percent of inhibition±SEM, *p<0.05 ANOVA.

FIG. 7 Anxiolytic effects of pridopidine in the rat ultrasonic vocalization (USV) test using pridopidine. USVs are considered a measure of anxiety in rodents. Young adult rats were treated with 30 mg/kg pridopidine by oral gavage (per os). Rats were first primed by a series of up to 10 electric shocks delivered to the feet by a grid in the floor, which was terminated in case of 3 consecutive and consistent USVs. The next day, each rat received 5 initial shocks, and USVs were recorded in the following 3-minute period. Animals were tested 30 and 120 minutes after pridopidine administration. Pridopidine significantly inhibited USV duration in rats. Data shown are mean USV time (sec)±SEM, N=4 rats per treatment group, *p<0.05.

FIG. 8 presents an improvement in the Problem Behaviors Assessment-Short (PBA-S) scale in HD patients treated with pridopidine in the Pride HD clinical trial, compared to placebo. PBA-S includes behavioral assessments of depressed mood, anxiety, apathy and irritability. Graph presents change from baseline, at week 52 in the placebo and 45 mg bid pridopidine treated groups.

FIG. 9 presents an improvement in the lack of initiative measure of the PBA-S in HD patients treated with pridopidine in the Pride-HD clinical trial, at week 52. The lack of initiative subscale of the PBA-S is a measure of apathy in HD patients. Apathy is a symptom common to HD and depression. Graph presents change from baseline, at week 52 in the placebo and 45 mg bid pridopidine treated groups.

FIG. 10 Study Schema of Phase 3, Randomized, Double-Blind Placebo-Controlled, clinical trial described in Example 3. (twice daily (bid); baseline (BL); end of study (EoS); early termination (ET); once daily (qd); visit (V); virtual visit (VV); week (W). * For each participant, the last treatment visit will be the EoS at either Week 65 or Week 78, if the participant completes all study visits, or Early Termination (ET) visit if the participant withdraws from the study before Week 65.).

FIG. 11 Study Schema—Open-Label Extension. (ET—early termination; V—visit; W—week).

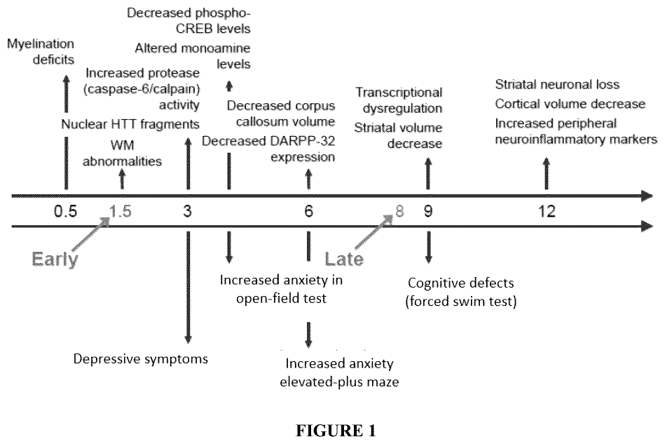

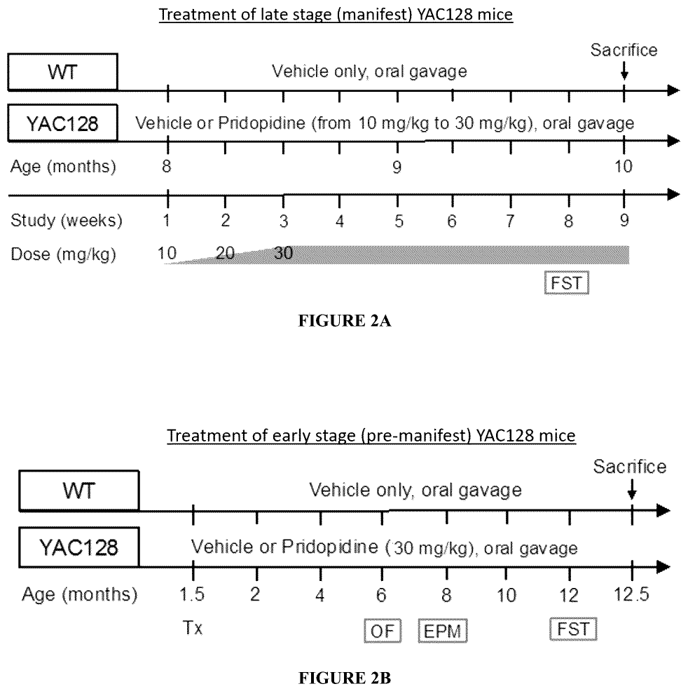

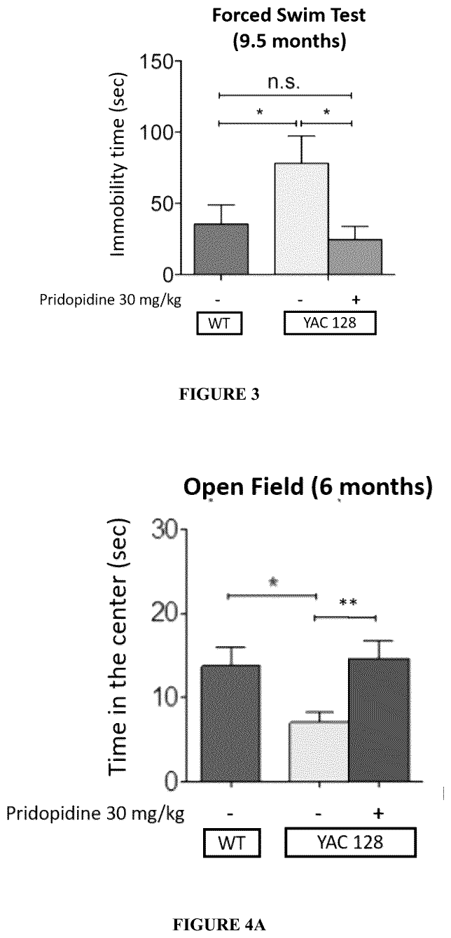

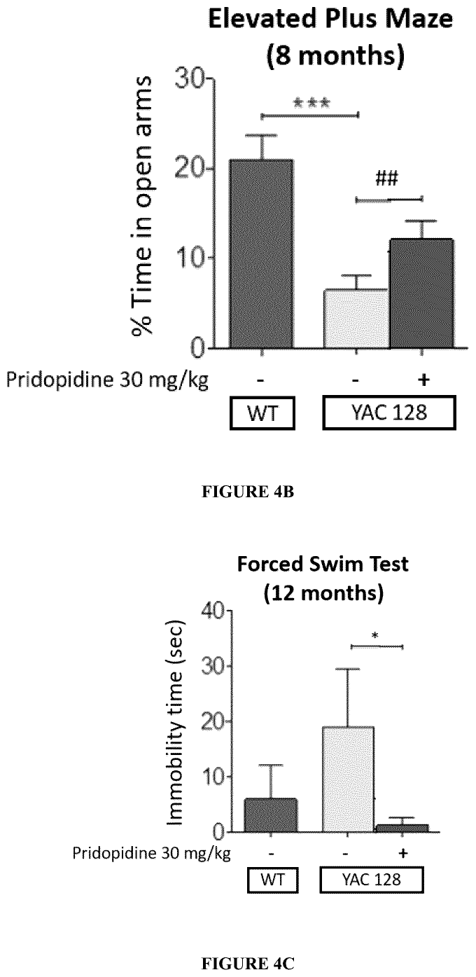

### DETAILED DESCRIPTION OF THE INVENTION

This invention provides a method of reducing anxiety and/or depression in a subject in need thereof comprising periodically administering to the subject a pharmaceutical composition comprising an amount of pridopidine effective to reduce anxiety and/or depression in a subject in the subject.

In one embodiment, the method reduces anxiety in the subject. In an embodiment, anxiety is measured by the State-Trait Anxiety Inventory (STAI), the problem Behaviors Assessment-Short (PBA-S) scale, the Fear Survey Schedule, Beck Anxiety Inventory (BAI), Brief Fear of Negative Evaluation Scale—BFNE, Clinician Administered PTSD Scale (CAPS), Daily Assessment of Symptoms—Anxiety, Generalized Anxiety Disorder 7 (GAD-7), Hamilton Anxiety Scale (HAM-A), Hospital Anxiety and Depression Scale (HADS-A), Leibowitz Social Anxiety Scale (LSAS), Overall Anxiety Severity and Impairment Scale (OASIS), Panic and Agoraphobia Scale (PAS), Panic Disorder Severity Scale (PDSS), PTSD Symptom Scale—Self-Report Version, Social Phobia Inventory (SPIN), Trauma Screening Questionnaire, Yale-Brown Obsessive Compulsive Scale (Y-BOCS), or the Zung Self-Rating Anxiety Scale.

In one embodiment, anxiety is reduced by at least one increment.

In another embodiment, the method reduces depression in the subject. In an embodiment, depression is measured by Hamilton Rating Scale for Depression (HAM-D), Beck Depression Inventory (BDI), Beck Hopelessness Scale, Centre for Epidemiological Studies-Depression Scale (CES-D), Patient Health Questionnaire, Center for Epidemiological Studies Depression Scale for Children (CES-DC), Clinically Useful Depression Outcome Scale, Diagnostic Inventory for Depression, Edinburgh Postnatal Depression Scale (EPDS), Inventory of Depressive Symptomatology, Geriatric Depression Scale (GDS), Hospital Anxiety and Depression Scale, Kutcher Adolescent Depression Scale (KADS), Major Depression Inventory (MDI), Montgomery-Asberg Depression Rating Scale (MADRS), Mood and Feelings Questionnaire (MFQ), Zung Self-Rating Depression Scale, or Cornell Scale for Depression in Dementia (CSDD).

In one embodiment, depression is reduced by at least one increment.

In another embodiment, the subject is afflicted with an anxiety disorder. In an embodiment, the anxiety disorder is generalized anxiety disorder (GAD), panic disorder, a phobic disorder, social phobia, agoraphobia, or trauma- and stressor-related disorders. In a further embodiment, the trauma- and stressor-related disorder is acute stress disorder (ASD), or posttraumatic stress disorder (PTSD).

In another embodiment, the subject is afflicted with a depressive disorder. In an embodiment, the depressive disorder is major depressive disorder, persistent depressive disorder, premenstrual dysphoric disorder, other depressive disorder, depressive disorder due to another medical condition, substance/medication-induced depressive disorder, perinatal depression, peripartum-onset depression, seasonal affective disorder, or psychotic depression.

In one embodiment, the subject is afflicted with a neurodegenerative disease. In another embodiment, the subject is afflicted with Huntington disease. In a further embodiment, the subject is afflicted with Stage 1 or Stage 2 Huntington disease. In one embodiment, the subject is afflicted with Stage 1 Huntington disease. In another embodiment, the subject is afflicted with Stage 2 Huntington disease.

In another embodiment, the subject is afflicted with early stage Huntington disease.

In an embodiment, the subject has greater than or equal to 36 CAG repeats in the huntingtin gene. In another embodiment, subject has greater than 44 CAG repeats in the huntingtin gene.

In some embodiments, the subject is presymptomatic. In other embodiments, the subject is symptomatic.

In one embodiment, the method comprises reducing anxiety in a subject afflicted with early stage Huntington disease.

In another embodiment, the method comprises reducing depression in a subject afflicted with any one of the following: Stage 1 Huntington disease, Stage 2 Huntington disease, Stage 3 Huntington disease, Stage 4 Huntington disease and Stage 5 Huntington disease.

In an embodiment, the subject is a human subject.

In an embodiment, the periodic administration is oral.

In an embodiment, between 22.5-315 mg pridopidine is administered to the subject per day. In another embodiment, 22.5 mg, 45 mg, 67.5, mg, 90 mg, 100 mg, 112.5 mg, 125 mg, 135 mg, 150 mg, 180 mg, 200 mg, 225mg, 250 mg, or 315 mg pridopidine is administered to the subject per day.

In an embodiment, the amount of pridopidine is administered by a unit dose of 22.5 mg, 45 mg, 67.5, mg, 90 mg, 100 mg, 112.5 mg, 125 mg, 135 mg, 150 mg, 180 mg, 200 mg, 225 mg, 250 mg, or 315 mg pridopidine.

In an embodiment, the unit dose is administered once daily.

In another embodiment, the pharmaceutical composition is administered for more than 26 weeks, at least 52 weeks, at least 54 weeks, at least 78 weeks, at least 104 weeks or more.

In an embodiment, the unit dose is administered more than once daily. In another embodiment, the unit dose is administered twice per day.

In an embodiment, the pridopidine is in the form of pridopidine hydrochloride.

In one embodiment, the anxiety and/or depression is reduced for at least 12 months.

The invention also provides pridopidine for use in reducing anxiety and/or depression in a subject.

In an embodiment, the subject has been diagnosed with anxiety only. In another embodiment, the subject is experiencing at least one symptom of anxiety, wherein the at least one symptom comprises restlessness, heart palpitations, hyperventilation, heavy sweating, muscle twitching, weakness, lethargy, insomnia, nausea, repetitive behavior, or any combination thereof.

In an embodiment, the subject has been diagnosed with depression only. In another embodiment the subject is experiencing at least one symptom of depression, and wherein the at least one symptom of depression comprises depressed mood, anhedonia, low energy levels, feelings of guilt, psychomotor retardation, agitation, suicidal ideations poor concentration and indecisiveness, or any combination thereof.

In an embodiment, a titration dose of an amount different from the intended dose is administered for a period of time at the start of the periodic administration. In some embodiments, the titration dose is half the amount of the intended dose. In another embodiment, the titration dose is administered in one administration per day and the intended dose is administered in two administrations per day. In one embodiment, the titration dose is administered for 7-21 or 7-14 days prior to the administration of the intended dose. In another embodiment, the titration dose is administered for 7 days, 14 days, or 21 days prior to the administration of the intended dose. The titration dose is preferably administered for fourteen days prior to the administration of the intended dose.

The invention also provides pridopidine for the manufacture of a medicament for use in reducing anxiety and/or depression in a subject.

The invention also provides a pharmaceutical composition comprising an effective amount of pridopidine for reducing anxiety and/or depression in a subject.

The invention also provides a pharmaceutical composition comprising pridopidine for use in reducing anxiety and/or depression a subject.

The invention also provides a package comprising:
a) a pharmaceutical composition comprising an amount of pridopidine; and
b) instructions for use of the pharmaceutical composition to reduce anxiety and/or depression in a subject.

The invention also provides a therapeutic package for dispensing to, or for use in dispensing to, a subject, which comprises:
a) one or more unit doses, each such unit dose comprising an amount of pridopidine thereof, wherein the amount of said pridopidine in said unit dose is effective, upon administration to said subject, to reduce anxiety and/or depression in the subject, and
b) a finished pharmaceutical container therefor, said container containing said unit dose or unit doses, said container further containing or comprising labeling directing the use of said package in the reduction of anxiety and/or depression in said subject.

The invention further provides a method of modulating gene expression in a subject afflicted with Huntington disease comprising administering an amount of pridopidine effective to modulate gene expression as described in this application.

The invention further provides a method of predicting clinical responsiveness to pridopidine therapy in a subject afflicted with Huntington disease, the method comprising evaluating expression of a biomarker in the subject, so as to thereby predict clinical responsiveness to pridopidine, wherein the biomarker is a gene as described in this application.

In one embodiment, the method further comprising predicting positive clinical responsiveness to pridopidine if the biomarker is up-regulated in the subject.

In another embodiment, the method further comprising predicting positive clinical responsiveness to pridopidine if the biomarker is suppressed in the subject.

In one embodiment, the subject is identified as a pridopidine responder if expression of the biomarker is higher than a reference value. In another embodiment, the subject is identified as a pridopidine responder if expression level of the biomarker is lower than a reference value.

In another embodiment, if the subject is identified as a pridopidine responder, the subject is thereafter administered a pharmaceutical composition comprising pridopidine.

Combinations of the above-described embodiments are also within the scope of the invention.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

### Terms

As used herein, and unless stated otherwise, each of the following terms shall have the definition set forth below.

As used herein, “administering to the subject” means the giving of, dispensing of, or application of medicines, drugs, or remedies to a subject to relieve, cure or reduce the symptoms associated with a disease, disorder or condition, e.g., a pathological condition. Oral administration is one way of administering the instant compounds to the subject.

As used herein, an “amount” or “dose” of pridopidine as measured in milligrams refers to the milligrams of pridopidine (4-[3-(methylsulfonyl)phenyl]-1-propyl-piperidine) present in a preparation, regardless of the form of the preparation. For example, a unit dose containing “90 mg pridopidine” means the amount of pridopidine base in a preparation is 90 mg, regardless of the form of the preparation. Thus, when in the form of a salt, e.g. pridopidine hydrochloride salt, the weight of the salt form necessary to provide a dose of 90 mg pridopidine would be greater than 90 mg due to the presence of the salt.

As used herein, a “unit dose”, “unit doses” and “unit dosage form(s)” mean a single drug administration entity/entities.

As used herein, “about” in the context of a numerical value or range means ±10% of the numerical value or range recited or claimed.

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat cognitive deficit. The specific effective amount varies with such factors as the particular condition being treated, the physical condition of the patient, the type of mammal being treated, the duration of the treatment, the nature of concurrent therapy (if any), and the specific formulations employed and the structure of the compounds or its derivatives.

As used herein, to “treat” or “treating” encompasses, e.g., inducing inhibition, regression, or stasis of a disorder and/or disease, e.g. depression, or alleviating, lessening, suppressing, inhibiting, reducing the severity of, eliminating or substantially eliminating, or ameliorating a symptom of the disease or disorder.

As used herein, “inhibition” of disease progression or disease complication in a subject means preventing, delaying or reducing the disease progression and/or disease complication in the subject. This includes, for example, delaying the progression of one of more symptoms in the subject, including but not limited to delaying the progression of: cognitive impairment, intellectual disability, learning disabilities (e.g., having difficulty learning new skills), developmental delays (e.g., not sitting, walking, or talking at the same time as other children the same age), social and behavior problems (e.g., making eye contact, anxiety, trouble paying attention, hand flapping, acting and speaking without thinking, and being very active), anxiety and hyperactive behavior, hypersensitivity to sensory stimuli, altered gastrointestinal function, autistic symptoms (e.g., shyness, poor eye contact, and social anxiety in mild cases to hand flapping, hand biting and preservative speech in the severely affected), attention deficit and hyperactivity, behavioral disturbances (e.g., irritability, aggression and self-injurious behaviors), seizures, obsessive-compulsive behavior and altered gastrointestinal function.

In some embodiments, symptoms of anxiety include but are not limited to restlessness, heart palpitations, hyperventilation, heavy sweating, muscle twitching, weakness, lethargy, insomnia, nausea, repetitive behavior, or any combination thereof.

In some embodiments, symptoms of depression include but are not limited to depressed mood, anhedonia, low energy levels, feelings of guilt, psychomotor retardation, agitation, suicidal ideations poor concentration, indecisiveness, or any combination thereof.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

As used herein, “pridopidine” means pridopidine base or a pharmaceutically acceptable salt thereof, or combination with pridopidine analogs or derivatives thereof for example deuterium-enriched version of pridopidine and salts. Examples of deuterium-enriched pridopidine and salts and their methods of preparation may be found in U.S. Application Publication Nos. 2013-0197031, 2016-0166559 and 2016-0095847, the entire content of each of which is hereby incorporated by reference. In certain embodiments, pridopidine is a pharmaceutically acceptable salt, such as the HCl salt or tartrate salt.

Preferably, in any embodiments of the invention as described herein, the pridopidine is in the form of its hydrochloride salt.

“Deuterium-enriched” means that the abundance of deuterium at any relevant site of the compound is more than the abundance of deuterium naturally occurring at that site in an amount of the compound. The naturally occurring distribution of deuterium is about 0.0156%. Thus, in a “deuterium-enriched” compound, the abundance of deuterium at any of its relevant sites is more than 0.0156% and can range from more than 0.0156% to 100%. Deuterium-enriched compounds may be obtained by exchanging hydrogen with deuterium or synthesizing the compound with deuterium-enriched starting materials.

A dosage unit can be prepared for oral dosage forms, such as tablets, capsules, pills, powders, and granules. A dosage unit can be prepared for intravenous dosage forms.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of the invention.

A “salt thereof” is a salt of the instant compound which has been modified by making acid or base salts of the compound. The term “pharmaceutically acceptable salt” in this respect, refers to the relatively non-toxic, inorganic and organic acid or base addition salts of compound of the present invention suitable for pharmaceutical use. Pharmaceutically acceptable salts may be formed by procedures well known and described in the art. One means of preparing such a salt is by treating a compound of the present invention with an inorganic base.

Examples of pharmaceutically acceptable salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

### Pridopidine Analogs

In some embodiments the methods of this invention make use of a pharmaceutical composition comprising pridopidine or pharmaceutically acceptable salt thereof and at least one analog thereof and pharmaceutically acceptable salt thereof. In another embodiment, the analog compounds of pridopidine or salts thereof are represented by the following structures of compounds 1-7:

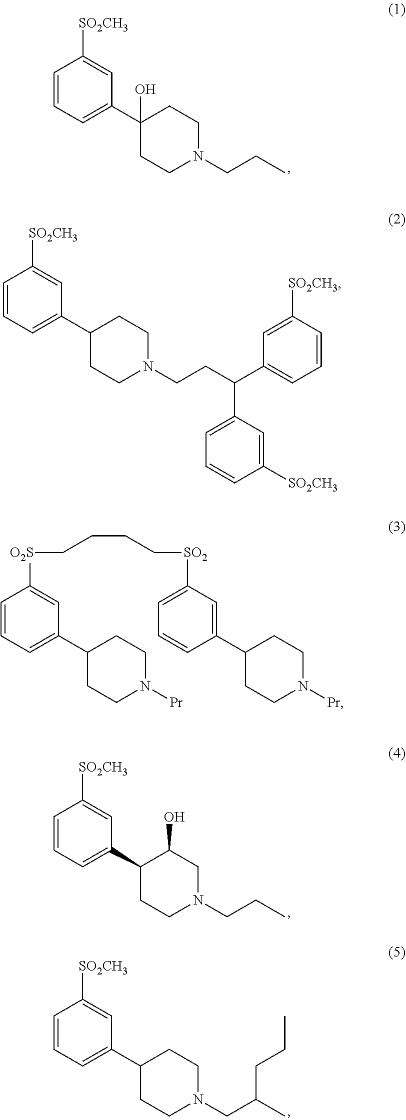

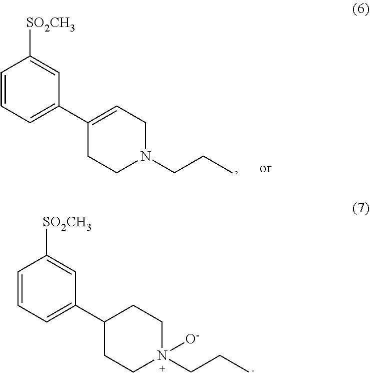

In other embodiments this invention provides a pharmaceutical composition comprising pridopidine or pharmaceutically acceptable salt thereof and compound 1 or pharmaceutically acceptable salt thereof. In other embodiments this invention provides a pharmaceutical composition comprising pridopidine or pharmaceutically acceptable salt thereof and compound 2 or pharmaceutically acceptable salt thereof. In other embodiments this invention provides a pharmaceutical composition comprising pridopidine or pharmaceutically acceptable salt thereof and compound 3 or pharmaceutically acceptable salt thereof. In other embodiments this invention provides a pharmaceutical composition comprising pridopidine or pharmaceutically acceptable salt thereof and compound 4 or pharmaceutically acceptable salt thereof. In other embodiments this invention provides a pharmaceutical composition comprising pridopidine or pharmaceutically acceptable salt thereof and compound 5 or pharmaceutically acceptable salt thereof. In other embodiments this invention provides a pharmaceutical composition comprising pridopidine or pharmaceutically acceptable salt thereof and compound 6 or pharmaceutically acceptable salt thereof. In other embodiments this invention provides a pharmaceutical composition comprising pridopidine or pharmaceutically acceptable salt thereof and compound 7 or pharmaceutically acceptable salt thereof. In other embodiments this invention provides a pharmaceutical composition comprising pridopidine or pharmaceutically acceptable salt thereof and compound 1 and compound 4 or pharmaceutically acceptable salt thereof. In other embodiments, the concentration of compounds 1, 2, 3, 4, 5, 6 or 7 or pharmaceutically acceptable salt thereof within the composition is between 0.001% w/w to 10% w/w. In other embodiments, the concentration of compounds 1, 2, 3, 4, 5, 6 or 7 or pharmaceutically acceptable salt thereof within the composition is between 0.001% w/w to 0.05% w/w. In other embodiments, the concentration of compounds 1, 2, 3, 4, 5, 6 or 7 or pharmaceutically acceptable salt thereof within the composition is between 0.001% w/w to 0.5% w/w. In other embodiments, the concentration of compounds 1, 2, 3, 4, 5, 6 or 7 or pharmaceutically acceptable salt thereof within the composition is between 0.001% w/w to 0.15% w/w. In other embodiments, the concentration of compounds 1, 2, 3, 4, 5, 6 or 7 or pharmaceutically acceptable salt thereof within the composition is between 0.01% w/w to 0.15% w/w. In other embodiments, the concentration of compounds 1, 2, 3, 4, 5, 6 or 7 or pharmaceutically acceptable salt thereof within the composition is between 0.01% w/w to 0. 5% w/w. In other embodiments, the concentration of compounds 1, 2, 3, 4, 5, 6 or 7 or pharmaceutically acceptable salt thereof within the composition is between 0.01% w/w to 1% w/w.

### Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

In an embodiment, the invention provides pharmaceutical compositions comprising the active compounds or pharmaceutically acceptable salts or derivatives thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, PA).

It is understood that where a parameter range is provided, all integers within that range, and tenths thereof, are also provided by the invention. For example, “22 mg-300.0 mg” includes 22.0, 22.1 mg, 22.2 mg, 22.3 mg, 22.4 mg, etc. up to 300.0 mg.

### Anxiety Rating Scales

The anxiety rating scales listed herein are known to those skilled in the art. For example, the Beck Anxiety Inventory (BAI) is a measure of anxiety that has 21 items which are summed to obtain a total score from 0-63, in which a score of 0-9 is generally considered to mean normal or no anxiety; a score of 10-18 is generally considered to mean mild to moderate anxiety; a score of 19-29 is generally considered to mean moderate to severe anxiety; and a score of 30-63 is generally considered to mean severe anxiety (Julian 2011). Another anxiety rating scale is the Hospital Anxiety and Depression Scale-Anxiety (HADS-A) which has 7 items (Julian 2011). This scale evaluates common dimensions of anxiety and can be used to detect and quantify magnitude of symptoms of anxiety (Julian 2011). The total score for HADS-A can range from 0 to 21 and the following guidelines are recommended for the interpretation of scores: 0-7 for normal or no anxiety, 8-10 for mild anxiety, 11-14 for moderate anxiety, and 12-21 for severe anxiety (Julian 2011). Other anxiety rating scales are described in Spitzer 2006, Hamilton 1959, Leary 1983, Heimberg 1999, Norman 2006, Zigmond 1983, and Connor 2000.

As used here, “reducing anxiety by at least one increment” means that the patient's anxiety as measured by at least one of the specific anxiety rating scales is lessened. For example, the STAI is an anxiety rating scale which has two subtest, a State Anxiety Scale (S-Anxiety) and a Trait Anxiety Scale (T-Anxiety) (Julian 2011). The range of scores for each subtest is 20-80 with a higher score indicating greater anxiety (Julian 2011). Therefore, a subject obtains a score of between 40 and 160 after completing the STAI. The subject's anxiety is reduced by at least one increment if the subject's STAI score is reduced by 1 or more points.

The patient anxiety may also be measured by one of the following anxiety rating scales: State-Trait Anxiety Inventory (STAI), the Fear Survey Schedule, Beck Anxiety Inventory (BAI), Brief Fear of Negative Evaluation Scale—BFNE, Clinician Administered PTSD Scale (CAPS), Daily Assessment of Symptoms—Anxiety, Generalized Anxiety Disorder 7 (GAD-7), Hamilton Anxiety Scale (HAM-A), Hospital Anxiety and Depression Scale (HADS-A), Leibowitz Social Anxiety Scale (LSAS), Overall Anxiety Severity and Impairment Scale (OASIS), Panic and Agoraphobia Scale (PAS), Panic Disorder Severity Scale (PDSS), PTSD Symptom Scale—Self-Report Version, Social Phobia Inventory (SPIN), Trauma Screening Questionnaire, Yale-Brown Obsessive Compulsive Scale (Y-BOCS), and the Zung Self-Rating Anxiety Scale.

### Depression Rating Scales

The depression rating scales listed herein are known to those skilled in the art. For example, Hamilton Depression Rating Scale (HAM-D) may be used to determine a patient's level of depression. The HAM-D lists 21 items, but scoring is based on the first 17 in which 0-6 is considered normal, 7-17 is considered mild depression, 18-24 is considered moderate depression, and 25 and greater is considered severe depression (Cusin 2009). Other depression rating scales are described in Bech 2001, Bech 2006, Strik 2001, and Cusin 2009.

As used here, “reducing depression by at least one increment” means that the patient's depression as measured by at least one of the specific depression rating scales is lessened. For example, in the HAM-D scale discussed above, the subject's depression is reduced by at least one increment if the subject's HAM-D score is reduced by 1 or more points.

The patient's depression may also be measured by one of the following depression rating scales: Hamilton Rating Scale for Depression (HAM-D), Beck Depression Inventory (BDI), Beck Hopelessness Scale, Centre for Epidemiological Studies-Depression Scale (CES-D), Patient Health Questionnaire, Center for Epidemiological Studies Depression Scale for Children (CES-DC), Clinically Useful Depression Outcome Scale, Diagnostic Inventory for Depression, Edinburgh Postnatal Depression Scale (EPDS), Inventory of Depressive Symptomatology, Geriatric Depression Scale (GDS), Hospital Anxiety and Depression Scale, Kutcher Adolescent Depression Scale (KADS), Major Depression Inventory (MDI), Montgomery-Asberg Depression Rating Scale (MADRS), Mood and Feelings Questionnaire (MFQ), Zung Self-Rating Depression Scale, or Cornell Scale for Depression in Dementia (CSDD).

### PBA-S-test

In another embodiment, PBA-S is a measure of depression and anxiety.

The Problem Behaviors Assessment for Huntington Disease—Short Form (PBA-s) is an interview designed specifically for rating the severity and frequency of behavioral abnormalities in HD. PBA-S includes behavioral assessments of depressed mood, anxiety, apathy and irritability. This scale is widely used in HD clinical trial and recommended as a measure of screening behavioral symptoms, including depression and anxiety (Mestre et al, MDS 2016).

Because of the prominence of psychiatric symptoms in HD, it was recommended that the PBA-s form be used in all HD studies with any need for behavioral assessment as a comprehensive screen for the most common psychiatric symptoms in HD (Craufurd 2001, Kingma 2008). The PBA-s also includes questions concerning suicidal behavior, a particular concern in HD. The PBA-s is based on the same set of core behavioral symptoms as the UHDRS Behavioral questions, which were used previously as the global psychiatric measure in most HD studies. The PBA-s has more detailed questions and more specific guidance on administration and scoring

The PBA-s is a brief semi-structured interview covering the most common behavioral and psychiatric manifestations of HD. The interview is not restricted to a single construct, but rather covers several broad symptom domains relevant to HD, comprising 11 items: low mood, suicidal ideation, anxiety, irritability, anger/aggressive behavior, loss of motivation, perseverative thinking or behavior, obsessive-compulsive behaviors, paranoid thinking, hallucinations, behavior suggestive of disorientation. Each symptom is rated for severity on a 5-point scale according to detailed scoring criteria which roughly correspond to the following: 0=“not at all”; 1=trivial; 2=mild; 3=moderate (disrupting everyday activities) and 4=severe or intolerable. Each symptom is also scored for frequency on a 5-point scale as follows: 0=symptom absent; 1=less than once weekly; 2=at least once a week; 3=most days (up to and including some part of everyday); and 4=all day, every day. Severity and frequency scores are multiplied to produce an overall ‘PBA score’ for each symptom.

The reliably of the PBA-S score to assess behavioral problems in HD patients was studied in 732 patients from the TRACK-HD observational study. This study provides strong evidence that the PBA-s is a reliable instrument for assessing depressed mood, anxiety and apathy behavioral problems in HD (Callaghan et al, J Neuropsychiatry Clin Neurosci 2015)

Reliability of the PBA-s to assess behavioral problems in HD was also investigated in 152 HD patients vs 56 healthy controls. HD patients portrayed more apathy, depression and irritability than controls. The study also concluded that the PBA-s is a reliable and sensitive instrument for the evaluation of apathy, depression and irritability (Kingma et al, Gen Hosp Psychiatry. 2008).

To aid in efficient development of new HD research studies, the National Institute of Neurological Disorders and Stroke (NINDS) published recommendations for measurement selection in HD. NINDS recommended the use of the PBA-s scale for behavioral assessments instead of the UHDRS Behavioral Exam because the PBA-s showed support for reliability and validity in HD as well as a sensitive measure for responsiveness to change in HD (Carlozzi et al, J Huntingtons Dis. 2014)

### Forced Swim Test (FST)-Test

Forced swim test (FST), is one of the most commonly used assays for the study of depressive-like behavior in rodents. When placing a rodent (mouse or rat) in a container filled with water, it will first make efforts to escape but eventually will exhibit immobility that is considered to reflect a measure of behavioral despair. This test has been extensively used because it involves the exposure of the animals to stress, which was shown to have a role in the tendency for major depression. Additionally, the FST has been shown to share some of the factors that are influenced or altered by depression in humans, including changes in food consumption, sleep abnormalities and drug-withdrawal-induced anhedonia. Moreover, its sensitivity to a broad range of antidepressant drugs that makes it a suitable screening test is one of the most important features leading to its high predictive validity.

### Marble Burying-Test

Marble burying test is an animal model used in scientific research to depict anxiety or obsessive-compulsive disorder (OCD) behavior. It is based on the observation that rats and mice will bury either harmful or harmless objects in their bedding. When rodents are put in a cage with marbles they will bury the marbles. This behavior is seen as anxiety related or OCD behavior. When the rodents are injected with drugs used to treat anxiety or OCD, the amount of marbles buried decreases. The test is also sensitive to antidepressant agents.

### Ultrasonic Vocalizations (UST)-Test

In neonatal mice ultrasonic vocalizations test have been studied both as an early communicative behavior of the pup-mother dyad and as a sign of an aversive affective state. Adult mice of both sexes produce complex ultrasonic vocalization patterns in different experimental/social contexts. All these vocalizations are becoming an increasingly valuable assay for behavioral phenotyping throughout the mouse life-span and alterations of the ultrasound patterns have been reported in several mouse models of neurodevelopmental disorders. This test is a reliable method for detecting anxiolytic properties of test compounds.

### EXPERIMENTAL DETAILS

### Example 1: Evaluation of Pridopidine in Transgenic YAC128 Mouse Model of Huntington Disease

Pridopidine is currently in clinical development for Huntington disease (HD) and ALS.

This study investigated the efficacy and mechanism of action of pridopidine using the transgenic YAC128 mouse model of HD (Garcia-Miralles 2016). Pridopidine was administered to animals starting at early (1.5 months of age) or late stages of disease (8 months of age). In the early treatment cohort, animals were divided into two groups receiving vehicle or 30 mg/kg of pridopidine for a period of 10.5 months. In the late cohort, animals were divided into two groups receiving either 0 mg/kg or an escalating dose of pridopidine (10 mg/kg in week 1, 20 mg/kg in week 2, and 30 mg/kg in weeks 3-8). Pridopidine treated animals were evaluated using a battery of behavioral tests. Analysis reveals that chronic treatment with pridopidine improves anxiety-like and depressive-like phenotypes in the YAC128 HD mice.

### Materials and Methods:

Animals. Male and female YAC128 HD mice (line 53) expressing a full-length human HTT (huntingtin) transgene with 128 CAG repeats, maintained on the FVB/N strain were used. Mice were bred at the Biological Resource Centre (Agency for Science, Technology and Research, ASTAR), and group-housed with littermates of mixed genotype. Animals were maintained under a 12-h light cycle (lights on at 09:00) in a clean facility, and given free access to food and water. Experiments were performed with the approval of the Institutional Animal Care and Use Committee at the Biomedical Sciences Institute (ASTAR) and in accordance with their approved guidelines.

Administration of pridopidine. Pridopidine was dissolved in sterile water. Pridopidine and vehicle were administered daily by oral gavage for five days/week for 10.5 months for the early treatment cohort and 8 weeks for the late treatment cohort. Mice received vehicle (sterile water) or 30 mg/kg of pridopidine at a volume of 4 mL/kg. Animals were weighed every two weeks to ensure the correct dose was maintained.

### Study Design.

Late treatment cohort: Pridopidine was administered to animals in advanced stages of disease (8 months of age). At this age, mice present striatal atrophy and profound behavioural deficits. Animals were divided into two groups receiving either 0 mg/kg or an escalating dose of pridopidine (10 mg/kg in week 1, 20 mg/kg in week 2, and 30 mg/kg in weeks 3-8). A forced swim test was executed at 9.5 months of age. Mice were sacrificed following completion of behavioural testing at 10 months of age.

Early treatment cohorts: Pridopidine was administered to animals (two cohorts) in the early stages of disease (1.5 months of age). Mice were divided into three groups. A group of YAC128 HD mice received pridopidine at a dose of 30 mg/kg, whereas the remaining groups, WT mice and YAC128 HD mice, received an equivalent volume of vehicle. One cohort was behaviourally tested every two months commencing at 2 months of age. Mice were tested for psychiatric function (open field, elevated plus maze, and forced swim test). Tests were conducted at a set time during the day, prior to drug administration.

Pridopidine was administered to animals starting at early (1.5 months of age) or late stages of disease (8 months of age) 5 days a week by oral gavage. Table 1 shows the treatment protocol for early stage disease cohort (1.5 mo old YAC128 HD mice). Table 1 shows the treatment protocol for late stage disease cohorts (8 mo old YAC128 HD mice).

TABLE 1
Early stage pridopidine treatment
Pridopidine early treatment groups

| | Pridopidine dose | | | |
|---|---|---|---|---|
| Genotype | (mg/kg) | Sex | Number | Total N/treatment |
| WT | 0 | F | 10 | 20 |
| | | M | 10 | |
| YAC128 | 0 | F | 10 | 20 |
| | | M | 10 | |
| YAC128 | 30 | F | 10 | 20 |
| | | M | 10 | |

TABLE 2
Late stage pridopidine treatment
Pridopidine late treatment groups

| | Pridopidine | | | | | Total |
|---|---|---|---|---|---|---|
| Genotype | dose (mg/kg) | | | Sex | Number | N/treatment |
| WT | 0 | 0 | 0 | F | 3 | 12 |
| | | | | M | 9 | |
| YAC128 | 0 | 0 | 0 | F | 3 | 9 |
| | | | | M | 9 | |
| YAC128 | 10 | 20 | 30 | F | 3 | 10 |
| | | | | M | 7 | |

### Tests of Affective Function

Anxiety-like behavioral tests. The open-field (OF) and elevated plus maze (EPM) tests are used to assess anxiety-like behavioral in rodents. The time spent in the center of the arena in the OF and the time spent in the open arms of the maze in the EPM are considered measures of anxiety-like behavior.

Depressive-like behavioral tests. The Porsolt forced-swim test (FST) is used to assess depressive-like behavior in rodents. The time spent immobile is considered a measure of depressive-like behavior. Immobility scores for each mouse were determined by manual scoring.

### Results

A 30 mg/kg dose of pridopidine showed beneficial effects on anxiety- and depressive-like behaviors in HD YAC128 mice. The improvements were seen throughout the disease course, with reduced anxiety-like phenotypes at 6 and 8 months of age, and amelioration of depressive-like behaviour at 12 months of age.

The improvements in behavioural outcomes are not likely to represent acute effects given pridopidine's short half-life and the fact that on test days, pridopidine was administered after the behavioural assays were completed. Furthermore, the effects appear to be HD-specific as no effects were seen in treated WT mice (data not shown). In contrast, when pridopidine was administered to mice at a later time point when the disease was clearly manifest, the functional benefits were limited, although improvements in depressive-like behaviour were noted.

These findings show that early administration of pridopidine exhibits anxiolytic and antidepressant properties in YAC128 HD mice, and therefore may be used for the treatment psychiatric symptoms. This data also shows that late administration of pridopidine in manifest YAC128 HD mice is efficacious to treat depressive-like phenotypes.

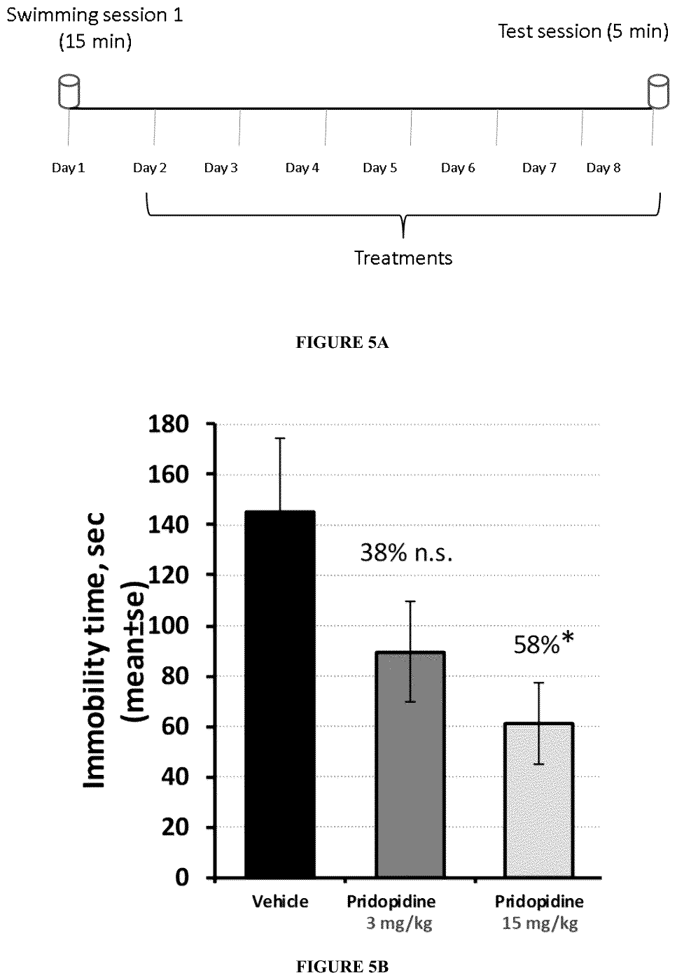

### Example 2: Anti-Depressive Effect in the Rat Forced Swim Test Using Pridopidine (FIGS. 5A and 5B).

Sprague-Dawley male rats, 6 weeks old were used. Rats were pre-tested on day 1 to ensure stable and high duration of immobility during the 5-min test session. Rats were then treated daily with pridopidine at 3 or 15 mg/kg by oral gavage for 7 days. On day 8, rats were administered the FST 30 minutes after pridopidine administration. Pridopidine decreased immobility time in rats by 38% and 58% in the 3 and 15 mg/kg groups, respectively (FIG. 5B). This indicates an anti-depressive effect of pridopidine in rats. This example demonstrates that pridopidine is a promising therapeutic target for depressive behavior.

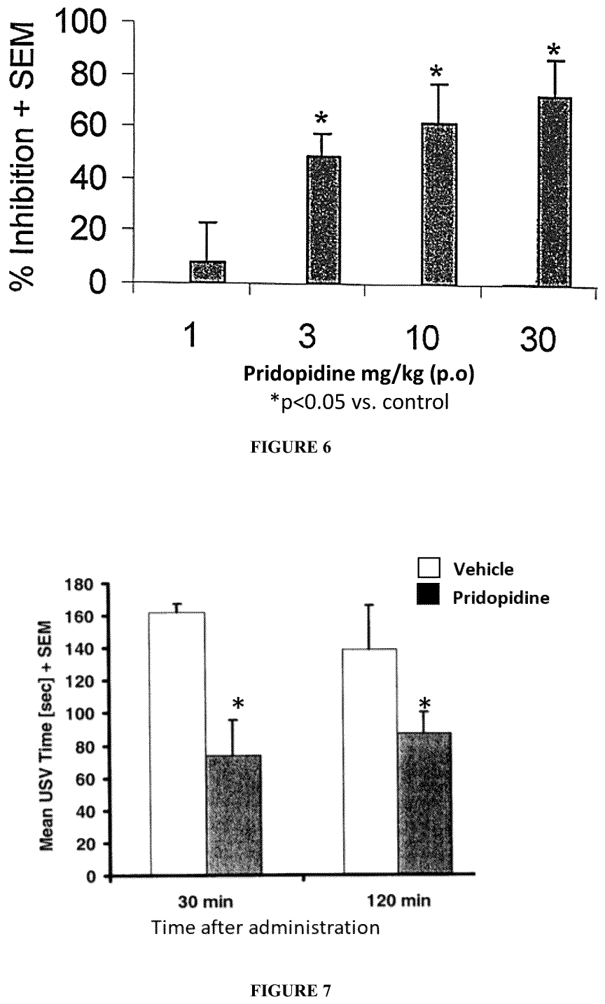

### Example 3: Pridopidine Shows an Anxiolytic Effect in the Marble Burying Test in Mice (NS) (FIG. 6).

Inhibition of object-burying in rodents is proposed as an animal model of anxiety because anxiolytic drugs reduce duration and extent of burying (Broekkamp et al, 1986; Treit, 1985; Treit et al, 1981).

### Materials and Methods

### Animals

Male NMRI mice (20-36 g body weight) were housed in groups of five in a temperature (20±2° C.) and humidity (50-60%) controlled colony room under anon-reversed 12 (6-18 on)/12 h light-dark cycle with food and water ad libitum except during the actual experiments. 8 mice were used for each drug dose and 16 mice for the vehicle-treated control group. Animals were assigned according to the experimental plan to one of the 4 test boxes run simultaneously.

### Procedure

30 min after drug application (pridopidine), mice were individually placed for 30 min in an open box (L 44 cm, W 43 cm, H 52 cm) filled with 5 cm of sawdust bedding material. 25 clean glass marbles (2 cm in diameter) were evenly spaced on the sawdust. The number of marbles covered by sawdust were counted. The experimenter also observed the animals for obvious inhibition of general activity (locomotor activity). Experiments were done between 8:30 and 12.00 a.m.

### Drugs

Pridopidine was suspended in 5% gum Arabic for p.o. application (application volume 1 ml/kg bodyweight).

### Results

Pridopidine treatment resulted in inhibition of marble-burying at all doses tested, in a dose-dependent manner (FIG. 6). 1 mg/kg demonstrated 8% inhibition; 3 mg/kg demonstrated 49% inhibition; 10 mg/kg demonstrated 62% inhibition; and 30 mg/kg demonstrated 73% inhibition.

Marble burying also represent an animal model for Obsessive-compulsive disorder.

### Example 4: Pridopidine Shows Anxiolytic Effects in the Rat Ultrasonic Vocalization (USV) Test (FIG. 7).

The ultrasonic vocalization (USV) test in young adult rats is one of the most robust animal models of anxiety amongst the various animal models used to detect anxiolytic-like effects in animals.

### Materials and Methods

### Animals

Male Sprague-Dawley rats (270-400 g bodyweight) were housed in groups of two in a temperature (20±2° C.) and humidity (50-60%) controlled colony room under a non-reversed 12 (6-18 on)/12 h light-dark cycle with food and water ad libitum except during the actual experiments. 4 rats were used for each drug dose and the vehicle-treated control group.

### Procedure

As described elsewhere (Bartoszyk G D (1998) Life Sci 22: 649-663; and Bartoszyk G D, (1997) Eur J Pharmacol 322: 147-153) USV was measured in a sound-attenuated test chamber (W 24 cm, L 22 cm, H 22 cm) with a grid floor for delivery of foot-shock (scrambled shock of 0.2 mA for 0.5 s). USV was recorded by a microphone and processed by an interface to select USV signals and to digitize the resulting signals for automatic processing. In the priming phase, each rat was placed in the test chamber. After a 2 min time period, a series of at most ten shocks (trials), 1.8 mA for 0.3 s, separated by 20 s shock-free intervals, was delivered via the grid floor of the test chamber. In the shock-free intervals the occurrence of ultrasonic vocalization (22±5 kHz) was automatically recorded, and the duration of ultrasonic vocalization was calculated immediately. The priming session was terminated either when the rat constantly vocalized at least for 10 s on three consecutive trials or after the tenth trial. Rats not responding with USV on three consecutive trials were excluded from the test. In the actual test performed on the next day, each rat received 5 initial shocks (1.8 mA for 0.3 s, separated by 20 s shock-free intervals) in the test chamber, and the duration of USV (22+4 kHz) was recorded during the following 3 min period. Animals were repeatedly tested 30 and 120 min after pridopidine administration.

### Drugs

Pridopidine was suspended in gum Arabic for p.o. application (application volume 10 ml/kg bodyweight).

### Results

Following oral administration, pridopidine 30 mg/kg significantly inhibited USV in young adult rats 30 min and 120 min after administration (FIG. 7).

### Example 5: Pridopidine is Effective in Non-HD Rodent Models of Anxiety

Pridopidine is administered periodically (e.g., daily or twice daily) to rodents exhibiting symptoms of anxiety. Examples of rodent models include the HAB rats, selected on the basis of their behavior in the elevated plus maze (EPM); the Syracuse High and Low Avoidance rats; the Maudsley reactive/nonreactive strains; the Tsukuba High and Low Emotional rats, and the Floripa H and L lines a rat model of anxiety and depression. The Roman Low-Avoidance (RLA) rats, selected on the basis of poor acquisition of a two-way avoidance response in the shuttle box, are considered as a model of high trait anxiety-emotionality. Selective breeding of rats and mice improves the probability of discovering anxiety-related neurobiological correlates, including genetic determinants, and allows the study of gene-environment interactions. (Steimer 2011).

Administering pridopidine is effective in treating anxiety. Administering pridopidine is effective in reducing symptoms of anxiety.

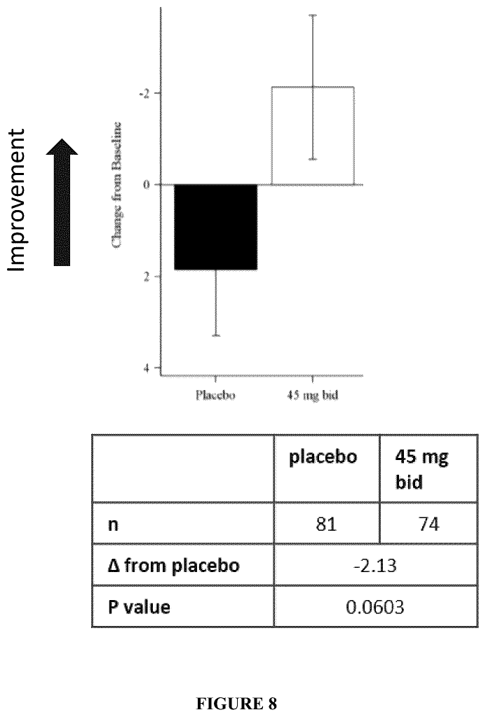

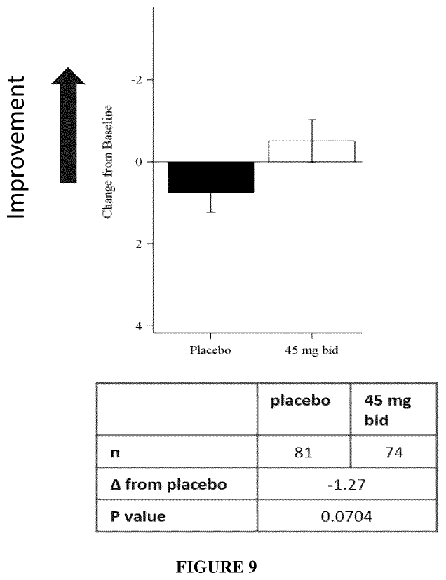

### Example 6: Pridopidine Improves Problem Behaviors Assessment-Short (PBA-S) in Pride-HD (FIGS. 8 and 9)

The Problem Behaviors Assessment-Short (PBA-s) scale, is a measure of psychiatric symptoms including anxiety, depression and apathy. The PBA-s is an interview designed specifically for rating the severity and frequency of behavioral abnormalities in HD. PBA-S includes behavioral assessments of depressed mood, anxiety, apathy and irritability. This scale is widely used in HD clinical trials and recommended as a measure of screening behavioral symptoms, including depression and anxiety. The effect of pridopidine on PBA-S in patients with HD in the Pride-HD clinical trial was assessed. After 52 weeks, a trend towards improvement of −2.13 units was demonstrated (p=0.0603) (FIG. 8). The lack of initiative subscale of the PBA-S is a measure of apathy in HD patients. Apathy is a symptom common to HD and depression. Pridopidine treatment demonstrates a trend towards improvement of −1.27 units was observed in the PBA-S lack of initiative measure (p=0.0704) (FIG. 9).

### Example 7: Assessment of Efficacy of Pridopidine in Treating Patients Suffering From Anxiety or Depression.

Pridopidine is administered periodically (e.g., daily or twice daily) to a patient diagnosed with anxiety or depression. The patient is exhibiting symptoms of anxiety or depression. The pridopidine is administered intravenously or orally. Administering pridopidine is effective in treating the patient. Administering pridopidine is also effective in reducing one or more of the symptoms of anxiety or of depression. Administering pridopidine is effective in facilitating rehabilitation of the patient.

Administering pridopidine is effective in facilitating rehabilitation of affective functions of the patient. Administering pridopidine is also effective in facilitating rehabilitation of behavioral function of the patient. Administering pridopidine is also effective in facilitating rehabilitation of emotional function of the patient. Administering pridopidine is also effective in facilitating rehabilitation of psychiatric function of the patient. Administering pridopidine is also effective in facilitating rehabilitation of sensory function of the patient.

### Example 8: A Phase III, A Randomized, Double-Blind, Placebo Controlled, Parallel Arm, Multicenter Study Evaluating the Efficacy and Safety of Pridopidine in Patients With Early Stage of Huntington Disease

### Objective

The proposed Phase 3 study is a 65 to 78-week, multicenter, randomized, double-blind, placebo controlled, parallel group study to evaluate the efficacy and safety of pridopidine administered at a dose of 45 mg bid in adult patients with early stage HD (TFC 7-13). Evaluation will be of total functional capacity, motor and behavioural features of HD in early-stage participants.

### Methods

The study consists of a screening period; a 2-week titration period; a 63-week, double-blind, full-dose treatment period; and a variable double-blind, full-dose treatment period up to 78 weeks, with a 2-week follow-up period.

Participants will be those with stage 1-2 HD, which is defined as a UHDRS-TFC score of ≥7, at screening Further, participants must have an UHDRS-Independence scale (IS) score of ≤90% at screening and a UHDRS-TMS≥20.

During the screening period, patients provide informed consent and subsequently undergo assessments to determine eligibility for participation in the study. The stage of HD is established by the UHDRS TFC scale. The TMS and UHDRS-IS are assessed.

Eligible patients are invited to return for a baseline visit and baseline assessments. Those patients who remain eligible for study participation will be randomly assigned (1:1 ratio) to 1 of the 2 treatment groups: 45 mg bid pridopidine or placebo bid. For patients assigned to receive pridopidine, the dose is titrated during the first 2 weeks from 45 mg qd to the final dose of 45 mg bid pridopidine.

### Overall Design of the Study:

The screening period will be followed by a 65 to 78 weeks double-blind treatment period, composed of a 2-week titration period, a 63 week double-blind full-dose maintenance treatment period followed by a variable double blind treatment period of up to 13 weeks (total of up to 78 weeks; Main study).

On Day 1 (Baseline visit), eligible participants will be randomized in a 1:1 ratio to active (pridopidine 45 mg bid) or control (placebo) ann.

Starting on Day 1, during the titration period, all participants will self-administer 1 capsule of study drug per os (PO-taken orally), once daily (qd), in the morning for 2 weeks. Thereafter, study drug will be taken PO bid in the morning and in the afternoon for 63 weeks (full-dose maintenance double-blind treatment period). Participants who complete the maintenance period (63 weeks) will continue into a variable double-blind period of up to 13 weeks or until the last participant randomized completes 65 weeks of treatment (2 weeks titration+63 weeks full dose), whichever comes first.

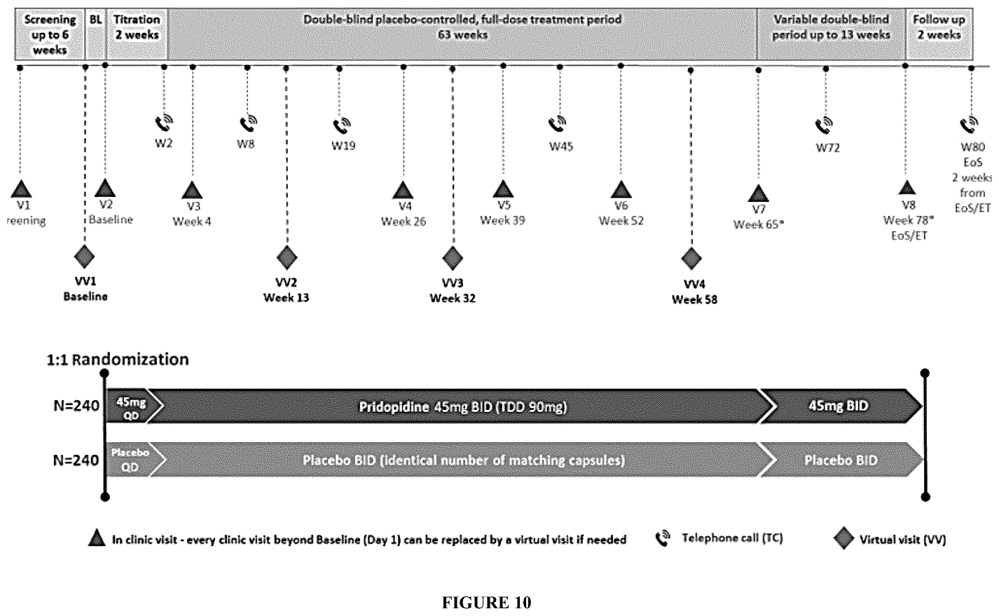

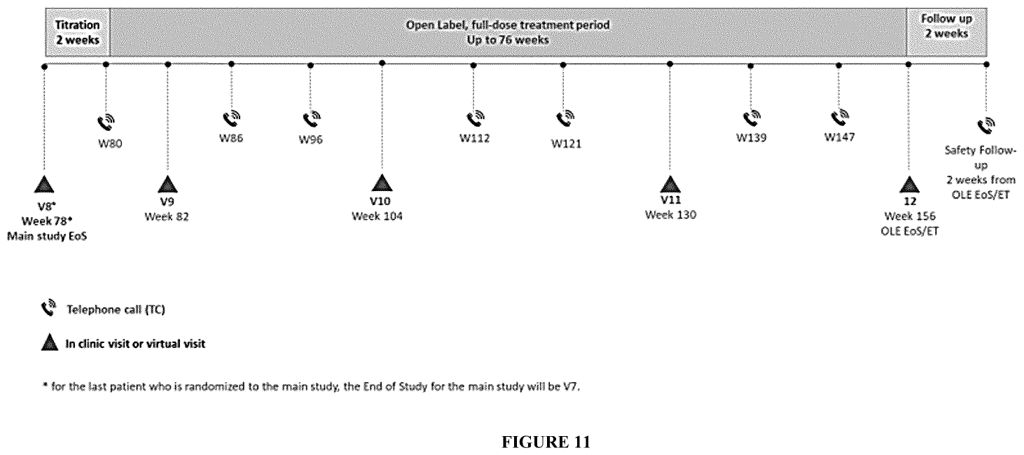

The Open Label Extension (OLE) will consist of a 2-week up titration period and a maintenance period. During the up-titration period, participants will self-administer 1 capsule of pridopidine 45 mg PO, qd, in the morning, for 2 weeks. Thereafter, pridopidine will be taken PO, bid in the morning and in the afternoon. Table 3 below presents the participants and study groups, FIG. 10 provides a Study Schema for the Main Study, and FIG. 11 provides the Study Schema for the Open-Label Extension (OLE).

TABLE 3
Treatment Groups - Main Study

| | Dose and dose regimen | | |
|---|---|---|---|
| | Titration Period | Maintenance Period | Number of |
| Treatment | (2 weeks) | (65 to 78 weeks) | participants |
| Active - pridopidine | 45 mg capsule | 45 mg capsule PO, | 240 |
| | PO, qd | bid (total daily | |
| | | dose of 90 mg) | |
| Control - matching | Capsule, PO, | Capsule, PO, bid | 240 |
| placebo | qd | | |

Pridopidine Dose Formulation, Route ofAdministration, Strength, and Levels

45 mg Pridopidine is provided in the form of a hard gelatin capsule for oral administration. The titration period includes administration of 45 mg capsule qd (on-prescription; morning dose) for 2 weeks, followed by the main full-dose treatment period wherein participants will take 45 mg capsule bid (1 capsule in the morning and 1 capsule in the afternoon, 7 to 10 hours after morning dose) for a total daily dose of 90 mg.

### Primary Endpoint

The primary efficacy endpoint to be evaluated is the change from baseline in UHDRS-TFC to week 65 in patients treated with pridopidine 45 mg bid compared to patients receiving placebo.

### Secondary and Exploratory Endpoints

Secondary endpoints will include: (a) Proportion of participants with no worsening (change≥0 point) from baseline to Week 65 in UHDRS-TFC (b) Change from baseline to Week 65 in the UHDRS-Total Motor Score (TMS) (c) Change from baseline to Week 65 in Quantitative motor (Q-Motor) finger tapping (Digitomotography) (d) Change from baseline to Week 65 in Composite UDEIRS (cUHDRS) total score (e) Change from baseline to Week 52 in UHDRS-TFC score (f) Change from baseline to Week 52 in UHDRS-TMS score (g) Proportion of participants with no worsening from baseline in Clinical Global Impression of Change (CGI-C) at Week 65

(h) Change from baseline to Week 78 in UHDRS-TFC score (i) Change from baseline to week 26, 52, 65 and 78 in PBA-s (j) Change from baseline to week 26, 52, 65 and 78 in PBA-s lack of initiative

The human patient's behavior and/or psychiatric state may be measured by the Problem Behaviors Assessment (PBA) total score. The human patient's behavior and/or psychiatric state may also be measured by the Problem Behaviors Assessment-short form (PBA-s). The human patient's behavior and/or psychiatric state may also be measured by the Problem Behaviors Assessment for depressed mood. The human patient's behavior and/or psychiatric state may also be measured by the Problem Behaviors Assessment for irritability. The human patient's behavior and/or psychiatric state may also be measured by the Problem Behaviors Assessment for lack of initiative or apathy. The human patient's behavior and/or psychiatric state may also be measured by the Problem Behaviors Assessment short form apathy sub-item. The human patient's behavior and/or psychiatric state may also be measured by the Apathy Evaluation Scale (AES). The human patient's behavior and/or psychiatric state may be measured by the Problem Behaviors Assessment for obsessive-compulsiveness. The human patient's behavior and/or psychiatric state may also be measured by the Problem Behaviors Assessment for disoriented behavior. In some embodiments, the human patient's behavior and/or psychiatric state is measured by the Problem Behaviors Assessment short form apathy sub-item or the Problem Behaviors Assessment-short form (PBA-s).

### REFERENCES

Bech P, et al. “The sensitivity and specificity of the Major Depression Inventory, using the Present State Examination as the index of diagnostic validity.”. J Affect Disord. 2001. 66: 159-64.

Bech P, “Rating scales in depression: limitations and pitfalls” Dialogues Clin Neurosci. 2006. 8(2): 207-215.

5 Bechtel, N. et al., Tapping linked to function and structure in premanifest and symptomatic Huntington disease. Neurology. 2010. 75(24):2150-60.

Benarroch E E. Effects of acetylcholine in the striatum. Recent insights and therapeutic implications. Neurology 2012;79(3):274-281.

Collaghan et al. Reliability and Factor Structure of the Short Problem Behaviors Assessment for Huntington's Disaese (PBA-s) in the TRACK-HD and REGISTRY studies. J. Neuropsychiatry Clin, Neurosci 2015 27(1): 59-64

Carlozzi et al. Understanding the outcome measures used in Huntington disease pharmacologicaltrials: A systemic review. J. Huntingtons Dis. 2014, 3(3): 233-252.

Carroll J B et al. Natural history of disease in the YAC128 mouse reveals a discrete signature of pathology in Huntington disease. Neurobiol. Dis. 2011.43(1):257-265.

Connor, K M, et al . Psychometric properties of the Social Phobia Inventory (SPIN) New self-rating scale. The British Journal of Psychiatry, 2000. 176(4), 379-386.

Craufurd D, et al. Behavioral changes in Huntington Disease. Neuropsychiatry Neuropsychol Behav Neurol. 2001. Oct-Dec;14(4):219-26.

Cusin, C, et al. Chapter 2: Rating Scales for Depression, L. Baer, M. A. Blais (eds.), Handbook of Clinical Rating Scales and Assessment in Psychiatry and Mental Health, Current Clinical Psychiatry.

Czeredys M, et al. Expression of genes encoding the calcium signalosome in cellular and transgenic models of Huntington's disease. Front Mol Neurosci 2013. 6:42.

Desplats P A et al. Glycolipid and ganglioside metabolism imbalances in Huntington's disease. Neurobiol. Dis. 2007. 27(3):265-277.

Fenster R J. Cell-Type Specific Translational Profiling in Huntington's Disease Mouse Models. Student Theses and Dissertations. Paper 148. 2011.

Garcia-Miralles M, et al. L6 Evaluation of pridopidine in the transgenic YAC128 mouse model of Huntington's disease. J Neurol Neurosurg Psychiatry 2016. 87:A92.

Geva , M. et al. Pridopidine activates neuroprotective pathways impaired in Huntington Disease. Human Molecular Genetics, 2016. 25(18):3975-3987

Hamilton M. “The assessment of anxiety states by rating.”. Br J Med Psychol. 1959 32 (1): 50-55.

Heimberg, RG, et al. Psychometric properties of the Liebowitz Social Anxiety Scale. Psychological medicine 1999. 29(1), 199.

Huntington Study Group. Unified Huntington's Disease Rating Scale: Reliablility and Consistency., Movement Disorders, 1996. 11(2):136-142.

Johnson A C and Paulsen J S. 2014. Huntington's Disease: A Guide for Professionals. D. Lovecky and K. Tarapata eds. Huntington's Disease Society of Americas (HDSA).

Julian, U. Measures of anxiety: State-Trait Anxiety Inventory (STAI), Beck Anxiety Inventory (BAI), and Hospital Anxiety and Depression Scale-Anxiety (HADS-A), Arthritis Care & Research November 2011.63:S11, S467—S472.

Kingma E M, et al. Behavioural problems in Huntington's disease using the Problem Behaviours Assessment. Gen Hosp Psychiatry. 2008. March-April; 30(2):155-6

Kuhn A, et al. Mutant huntingtin's effects on striatal gene expression in mice recapitulate changes observed in human Huntington's disease brain and do not differ with mutant huntingtin length or wild-type huntingtin dosage. Hum Mol Genet 2007. 16(15):1845-1861.

Kuleshov M V et al. Enrichr: a comprehensive gene set enrichment analysis web server 2016 update. Nucleic Acids Research 2016. 44(W1):W90-7.

Leary, M R. A brief version of the Fear of Negative Evaluation Scale. Personality and Social Psychology Bulletin, 1983. 9(3), 371-375.

Mestre et al., Rating Scales for Behavioral Symptoms in Huntington's Disease: Critique and Recommendations. Movement Disorders, 2016, 31 (10), 1466-1478.

Mineur, Y S. et al. “Cholinergic Signaling in the Hippocampus Regulates Social Stress Resilience and Anxiety- and Depression-like Behavior.” PNAS USA 2013.110(9): 3573-3578.

Norman, S B, et al. Development and validation of an overall anxiety severity and impairment scale (OASIS). Depression and anxiety, 2006. 23(4), 245-249.

Pisani A, et al. Re-emergence of striatal cholinergic interneurons in movement disorders. Trends Neurosci 2007. 30(10):545-553.

Ponten H, et al. In vivo pharmacology of the dopaminergic stabilizer pridopidine. Eur J Pharmacol. 2010. 644(1-3):88-95.

Pouladi M A et al. Marked differences in neurochemistry and aggregates despite similar behavioural and neuropathological features of Huntington disease in the full-length BACHD and YAC128 mice. Hum Mol Genet 2012. 21(10):2219-2232.

Shema R et al. Synthetic lethal screening in the mammalian central nervous system identifies Gpx6 as a modulator of Huntington's disease. PNAS 2014. 112(1):268-272.

Slow E J et al. Selective striatal neuronal loss in a YAC128 mouse model of Huntington disease. Hum Mol Genet 2003. 12(13):1555-1567.

Southwell A L et al. Anti-semaphorin 4D immunotherapy ameliorates neuropathology and some cognitive impairment in the YAC128 mouse model of Huntington disease. Neurobiol. Dis. 2015. 76:46-56.

Spitzer, R L, et al. A brief measure for assessing generalized anxiety disorder: the GAD-7. Archives of internal medicine, 2006. 166(10), 1092.

Steimer T. Animal models of anxiety disorders in rats and mice: some conceptual issues. Dialogues in Clinical Neuroscience. 2011.13(4):495-506.

Strik J, et al. Sensitivity and specificity of observer and self-report questionnaires in major and minor depression following myocardial infarction. Psychosomatics. 2001. 42:423-428.

Teo R T Y et al. Structural and molecular myelination deficits occur prior to neuronal loss in the YAC128 and BACHD models of Huntington disease. Hum Mol Genet 2016. 25(13):2621-2632.

van Roon-Mom W M C et al. Mutant huntingtin activates Nrf2-responsive genes and impairs dopamine synthesis in a PC12 model of Huntington's disease. BMC Mol Biol 2008. 9:84.

van Waarde A et al. The cholinergic system, sigma-1 receptors and cognition. Behav Brain Res 2011. 221(2):543-554.

Xie, Y, et al. BDNF Overexpression in the Forebrain Rescues Huntington's Disease Phenotypes in YAC128 Mice. J Neurosci. 2010. 30(44): 14708-14718.

Zigmond, A S, and Snaith, R P. The hospital anxiety and depression scale. Acta psychiatrica scandinavica, 1983. 67(6), 361-370.

CSID: 25948790, www.chemspider.com/Chemical-Structure.25948790.html (accessed 23:27, Jul. 15, 2016).

CSID: 7971505, www.chemspider.com/Chemical-Structure.7971505.html (accessed 23:33, Jul. 15, 2016).

U.S. Pat. No. 6,903,120, issued Jun. 7, 2005 (Sonesson et al.).

U.S. Pat. No. 7,923,459, issued Apr. 12, 2011 (Gauthier et al.).

US Patent Publication No. US2016/243098 published Aug. 25, 2016 (Geva et al).

What is claimed is:

1. A method of reducing depression in a subject with a depressive disorder comprising administering to the subject a pharmaceutical composition comprising an amount of pridopidine or pharmaceutically acceptable salt thereof; wherein the depressive disorder is major depressive disorder, persistent depressive disorder, premenstrual dysphoric disorder, substance/medication-induced depressive disorder, perinatal depression, peripartum-onset depression, seasonal affective disorder, or psychotic depression.

2. The method of claim 1, wherein the depression is reduced by at least one unit.

3. The method of claim 1, wherein the depression is measured by Hamilton Rating Scale for Depression (HAM-D), Beck Depression Inventory (BDI), Beck Hopelessness Scale, Centre for Epidemiological Studies-Depression Scale (CES-D), Patient Health Questionnaire, Center for Epidemiological Studies Depression Scale for Children (CES-DC), Clinically Useful Depression Outcome Scale, Diagnostic Inventory for Depression, Edinburgh Postnatal Depression Scale (EPDS), Inventory of Depressive Symptomatology, Geriatric Depression Scale (GDS), Hospital Anxiety and Depression Scale, Kutcher Adolescent Depression Scale (KADS), Major Depression Inventory (MDI), Montgomery-Asberg Depression Rating Scale (MADRS), Mood and Feelings Questionnaire (MFQ), Zung Self-Rating Depression Scale, forced swim test, or Cornell Scale for Depression in Dementia (CSDD).

4. The method of claim 1, wherein the subject is experiencing at least one symptom of depression, and wherein the at least one symptom of depression comprises depressed mood, anhedonia, low energy levels, feelings of guilt, psychomotor retardation, agitation, suicidal ideations poor concentration and indecisiveness, or any combination thereof

5. The method of claim 1, wherein between 22.5-315 mg pridopidine or corresponding amount of its pharmaceutically acceptable salt is administered to the subject per day.

6. The method of claim 1, wherein the unit dose is administered once daily, or more than once daily.

7. The method of claim 1, wherein the unit dose is administered twice per day.

8. The method of claim 1, wherein the administration is oral.

9. The method of claim 1, wherein the pridopidine is in the form of pridopidine hydrochloride.

10. The method of claim 1, wherein the depression is reduced for at least 12 months.
